(19) United States
(12) Patent Application Publication
(10) Pub. No.: US 2004/0111032 A1
(43) Pub. Date: Jun. 10, 2004

(54) Optical coupler for rotating catheter

(76) Inventor: Jeff Korn, Lexington, MA (US)

Correspondence Address: J. PETER FASSE, Fish & Richardson P.C., 225 Franklin Street, Boston, MA, 02110-2804, US

(21) Appl. No.: 10/309,477

(22) Filed: Dec. 4, 2002

(51) Int. Cl. A61B 6/00

(52) U.S. Cl. 600/478

(57) ABSTRACT

An optical coupler guides one or more rotating second beams from an eccentric port to a fixed point on a detector. The coupler includes a housing having a distal face with an eccentric port. A central relay located inside the housing guides the rotating second beams to the fixed point on the detector.

### TECHNICAL FIELD

This invention relates to fiber optic catheters, and more particularly to catheters that accommodate more than one optical fiber.

### BACKGROUND

Vulnerable plaques are lipid filled cavities that form within the wall of a blood vessel. These plaques, when ruptured, can cause massive clotting in the vessel. The resultant clot can interfere with blood flow to the brain, resulting in a stroke, or with blood flow to the coronary vessels, resulting in a heart attack.

To locate vulnerable plaques, one inserts a catheter through the lumen of the vessel. The catheter includes a delivery fiber for illuminating a spot on the vessel wall and one or more collection fibers for collecting scattered light from corresponding collection spots on the vessel wall. The delivery fiber, and each of the collection fibers form distinct optical channels within the catheter. The catheter used for locating plaques is thus a multi-channel catheter.

In operation, a light source outside the catheter introduces light into the delivery fiber. A detector, also outside the catheter, detects light in the collection fiber and generates an electrical signal representative of that light. This signal is then digitized and provided to a processor for analysis.

A vulnerable plaque can be anywhere within the wall of the artery. As a result, it is desirable to circumferentially scan the illuminated spot and the collection spot around the vessel wall. One way to do this is to spin the multi-channel catheter about its axis. However, since neither the light source nor the processor spin with the catheter, it becomes more difficult to couple light into and out of the delivery and collection fibers while the catheter is spinning.

### SUMMARY

The described device, method and system are based on the recognition that a lens can be made to focus light onto a fixed point even as the source of that light moves relative to the lens.

In one aspect, the invention includes an optical coupler having a housing with a distal face and an eccentric port. A central relay guides a beam from the eccentric port to a fixed point on a detector.

A first optical relay can be configured for guiding a first beam to a central port in the distal face. A second optical relay can be located between the central relay and the eccentric port for focusing the circularly rotating second beam from the rotating eccentric port to the central relay. The central relay can also be configured for guiding the circularly rotating second beam from the second optical relay to the fixed point on the detector. A source for the first beam can be located between the central relay and the central port. The central relay can have a lens having a central axis and a focal point, the focal point being radially displaced from the central axis. The second optical relay can also rotate with the second beam.

In another aspect, the invention features a central relay with walls forming an aperture disposed to allow passage of the first beam. The housing can have a proximal wall forming an aperture disposed to intersect the first beam. The housing can also have an additional optical relay disposed to guide the first beam, through the aperture, to the first optical relay

In another aspect, the invention features a central relay having a beam-directing element having a multiplicity of zones, each of which is configured to direct a beam incident thereon to the fixed point on the detector. The central relay could also be a lens with a multiplicity of regions, each region having a refractive index selected to refract a beam incident thereon to the fixed point on the detector. The central relay could also be a lens with a multiplicity of regions, each region having a radius of curvature selected to refract a beam incident thereon to the fixed point on the detector.

In another aspect, the invention features a third optical relay for guiding a rotating third beam from a second rotating eccentric port on the distal face to the central relay. In this case, the central relay is configured to guide the rotating third beam to a second fixed point on a second detector. The additional optical relay can have a graded index of refraction lens seated in the central port. The graded index of refraction lens is configured to direct the first beam to the central port. The second optical relay can have a collimating lens within the housing. In this case, the collimating lens can be disposed to guide the second beam from the eccentric port toward the central relay. The stationary relay also can have a light-directing element disposed to direct the second beam toward a peripheral wall of the housing.

Some embodiments of the invention include a first optical relay having a stationary lens to direct the first beam through the aperture. In some of these embodiments, a focusing lens is disposed between the aperture in the R-S lens and the central port. In yet other embodiments, the R-S lens and optical relays could include a variety of optical elements, such as graded index of refraction (“GRIN”) lenses, prisms, diffractive elements, and mirrors, arranged to direct light from a source to a destination

The invention includes embodiments that feature variations of the R-S lens. Among these are embodiments in which the center of the R-S lens is removed. In addition, the edge of the R-S lens can be reduced to only the portion of the lens through which the collection beam passes, thereby reducing the material required to construct the lens.

Additional variations of the second optical relay are those found in embodiments featuring one or more eccentric apertures in the distal face of the housing. These eccentric apertures allow passage of one or more corresponding second beams. These beams trace circular paths on the R-S lens, which then directs the beams to one or more stationary detectors. Also, these beam have differing central angles with respect to each other.

Another aspect of the invention is a system for identifying vulnerable plaque. In one embodiment, the system includes a catheter having a collection fiber and a delivery fiber extending through the catheter. The catheter engages a distal face of a stationary housing configured to couple the rotating catheter.

In an additional aspect, the invention provides a way to optically couple to a collection fiber and a delivery fiber. In one practice, the method includes transmitting a delivery beam through an aperture of a housing and guiding the delivery beam from the aperture to a central port in the housing, the central port being in optical communication with the delivery fiber. A collection beam is then received from an eccentric port in the housing, the eccentric port being in optical communication with the collection fiber. The collection beam is then guided to the detector, such that the focused spot on the detector is stationary as the collection fiber is revolved.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Embodiments of the invention may have one or more of the following advantages. By providing a continuous connection to both optical fibers, the rotary coupler permits the entire circumference of an artery to be scanned automatically.

A rotary coupler having the features of the invention can also be used to identify other structures outside but near a lumen, or on the surface of the lumen wall. For example cancerous growths within polyps can be identified by a catheter circumferentially scanning the lumen wall of the large intestine, cancerous tissue in the prostate may be identified by a catheter scanning the lumen wall of the urethra in the vicinity of the prostate gland, or Barrett's cells can be identified on the wall of the esophagus. In addition to its medical applications, the rotary coupler can be used in industrial applications to identify otherwise inaccessible structures outside pipes.

Other features and advantages of the invention will be apparent from the following detailed description, and from the claims.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a system for identifying vulnerable plaque in a patient.

FIG. 2 is a cross-section of the multi-channel catheter in FIG. 1.

FIG. 3 is a profile view of the multi-channel coupler of FIG. 1.

FIG. 4 is an end view of the multi-channel coupler of FIG. 1.

FIG. 5 is the same profile view of FIG. 3 with the core rotated 180 degrees.

FIG. 6 is a profile view of the multi-channel coupler incorporating additional fibers Like reference symbols in the various drawings indicate like elements.

### DETAILED DESCRIPTION

System Overview

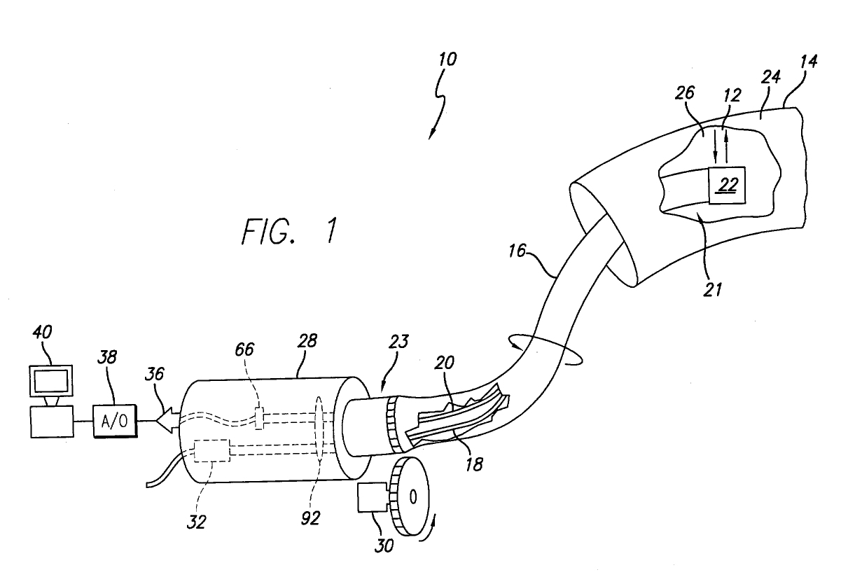

FIG. 1 shows a diagnostic system 10 for identifying vulnerable plaque 12 in an arterial wall 14 of a patient. The diagnostic system features a catheter 16 to be inserted into a selected artery, e. g. a coronary artery, of the patient. A delivery fiber 18 and a collection fiber 20 extend between a distal end 21 and a proximal end 23 of the catheter 16.

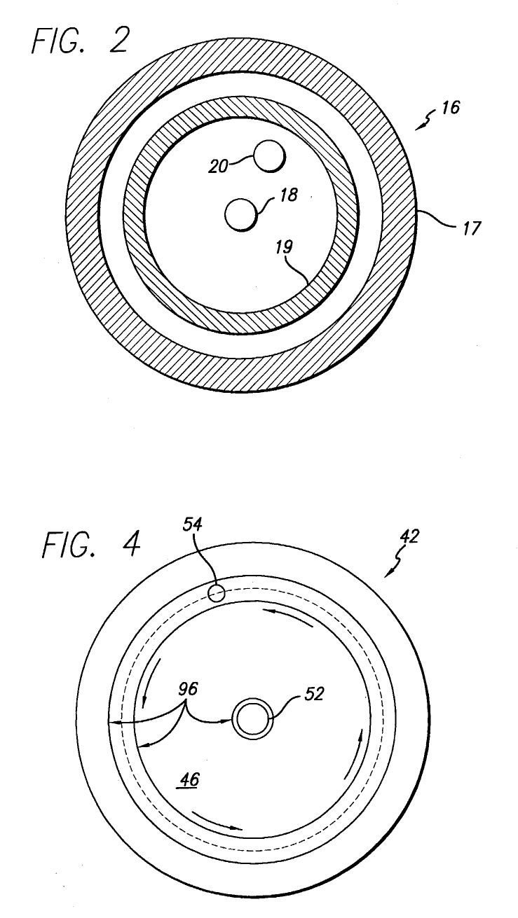

As shown in FIG. 2, the catheter 16 includes ajacket 17 surrounding a rotatable core 19. The delivery fiber 18 extends along the center of the core 19 and the collection fiber 20 extends parallel to, but radially displaced from, the delivery fiber 18. The rotatable core 19 spins at rate between approximately 4 revolutions per second and 30 revolutions per second.

Referring again to FIG. 1, at the distal end 21 of the catheter 16, a tip assembly 22 directs light traveling axially on the delivery fiber 18 toward an illumination spot 24 on the arterial wall 14. The tip assembly 22 also collects light from a collection spot 26 on the arterial wall 14 and directs that light into the collection fiber 20.

A multi-channel coupler 28, which is driven by a motor 30, engages the proximal end 23 of the catheter 16. The motor 30 spins the catheter 16, enabling the diagnostic system 10 to circumferentially scan the arterial wall 14 with the illumination spot 24.

The multi-channel coupler 28 guides a beam from a laser 32 (or other source, such as an LED, a super luminescent LED, or an arc lamp) into the delivery fiber 18 and guides light emerging from the collection fiber 20 into one or more detectors 66. The multi-channel coupler 28 performs these tasks while the catheter core 19 continuously spins.

The detectors provide an electrical signal indicative of light intensity to an amplifier 36 connected to an analog-to-digital (“A/D”) converter 38. The A/D converter 38 converts this signal into data that can be analyzed by a processor 40 to identify the presence of a vulnerable plaque 12 hidden beneath the arterial wall 14.

Coupler Rotary Junction to Catheter

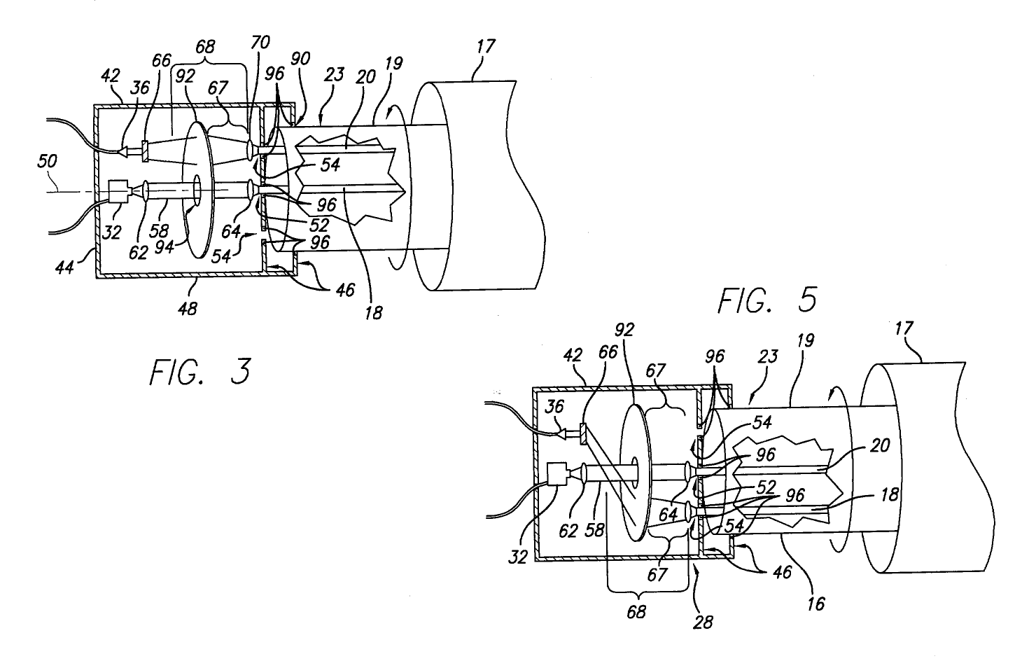

A multi-channel coupler 28 for carrying out the foregoing tasks, as shown in FIG. 3, includes a cylindrical housing 42 having a proximal face 44 joined to a distal face 46 by a peripheral wall 48. The distal face 46 of the housing 42 has a catheter core port 53 for receiving the catheter core 19, a central port 52 for receiving the delivery fiber 18, and an eccentric port 54 for receiving the collection fiber 20. The central port 52 is located at the intersection of the axis 50 with the distal face 46. The eccentric port .54 is radially displaced from the central port 52. As a result, when the catheter core 19 spins about the axis 50, the delivery fiber 18 remains stationary and the collection fiber 20 traces out a circular path, as shown in an end view in FIG. 4. Bearings 96 at the central port 52, eccentric port 54, and catheter core port 53 couple the housing 42 to the catheter core 19. The bearings 96 also enable the catheter core 19 to spin about an axis 50 that intersects the proximal and distal faces 44, 46 of the housing 42.

The distal face 46 of the housing 42 is rotatably coupled to the catheter 16. Two optical fibers extend through the catheter 16: a delivery fiber 18 for illuminating the arterial wall 14 and a collection fiber 20 that collects light scattered from the arterial wall 14. The catheter core 19 spins about axis 50 while the housing 42 centered about axis 50 remains stationary.

A laser 32 directed towards the distal face 46 produces a delivery beam 58 centered on the axis 50 as shown in FIG.3. A first collimating lens 62 collimates the delivery beam 58 and directs it through the housing 42 and through an aperture 94 of a rotation-to-stationary (R-S) lens 92. The R-S lens aperture 94 is a circular opening that is centered on the axis 50 and has a diameter slightly larger than the delivery beam 58. A first optical relay 64 within the housing 42 then receives the collimated delivery beam 58 and directs it distally across the housing 42 toward the central port 52, where it enters the delivery fiber 18. As used herein, an optical relay refers to a set of optical elements, such as lenses, prisms, and mirrors, arranged to direct light from a source to a destination.

In FIG. 3, this first optical relay 64 includes a converging lens focused at the central port 52. However, the first optical relay 64 can include components other than, or in addition to that shown in FIG. 3. Between the proximal face 44 and the central port 52, the delivery beam 58 is not constrained to travel along the axis 50 as shown in FIG. 3. The delivery beam 58 may travel on any path that leads to the delivery fiber 18. One design option, shown in FIG. 7, includes locating the laser 32 or directing the delivery beam 58 to start between the R-S lens 92 and the distal face 46. This would eliminate the need for the R-S lens aperture 94.

Between the proximal face 44 and the R-S lens 92 is a detector 66 for receiving a collection beam 68 entering through the eccentric port 54. The collection beam 67 is divided into a proximal side extending from the detector 66 to the R-S lens 92 and a distal side 67 extending from the R-S lens 92 to the eccentric port 54. A second optical relay 70 receives the collection beam 68 from the eccentric port 54 and directs it to the R-S lens 92. The R-S lens 92 directs the collection beam 68 to the detector 66 located towards the proximal face 44. The second optical relay 70 and the distal side of the collection beam 67 rotate circularly about the axis 50 and trace a circular path on the R-S lens 92. Without itself moving, the R-S lens 92 continuously redirects the collection beam 68 onto the stationary detector 66. In FIG. 5, the second optical relay 70 and the distal side of the collection beam 67 have rotated 180 degrees from the position depicted in FIG. 3. The R-S lens 92 directs the distal side of the collection beam 67, now located 180 degrees from its position in FIG. 3, back to the stationary detector 66 regardless of where the proximal side of the collection beam 67 intersects the R-S lens 92. The R-S lens 92 continuously directs the collection beam 68 onto the stationary detector 66 as the rotation of the core causes the optical relay and the distal side of the collection beam 67 to traverse a circular path on the R-S lens 92.

The geometry or grading index of the R-S lens 92 is not symmetric about the axis 50. Instead, the geometry or grading index of the R-S lens 92 varies as a function of angle. For example, the portion of the lens through which the collection beam 68 passes in FIG. 3 refracts the collection beam 68 less than the portion of the lens through which the collection beam 68 passes in FIG. 5. As a result, the R-S lens 92 redirects the distal side of the collection beam 67 to the stationary detector 66 even as the proximal side of the collection beam 67 traces a circular path on R-S lens 92. The R-S lens 92 can include a variety of optical elements, such as lenses, prisms, and mirrors, arranged to direct light from a rotating source to a fixed destination. A central portion of the lens can be removed or made transparent to allow the delivery beam 58 to pass unaltered. A peripheral portion of the R-S lens 92 can be reduced to only the portion of the lens through which the collection beam 68 passes, thereby forming a donut shaped lens. This donut shaped lens would reduce the material needed to produce the R-S lens 92.

### Other Embodiments

The optical couplers shown in FIGS. 1-5 are two-channel couplers. Each has a delivery channel that carries the delivery beam 58 and a collection channel for carrying a collection beam 68. However, additional collection or delivery channels can be added by providing additional collection ports or delivery ports, each of which is in communication with an additional collection fiber or delivery fiber.

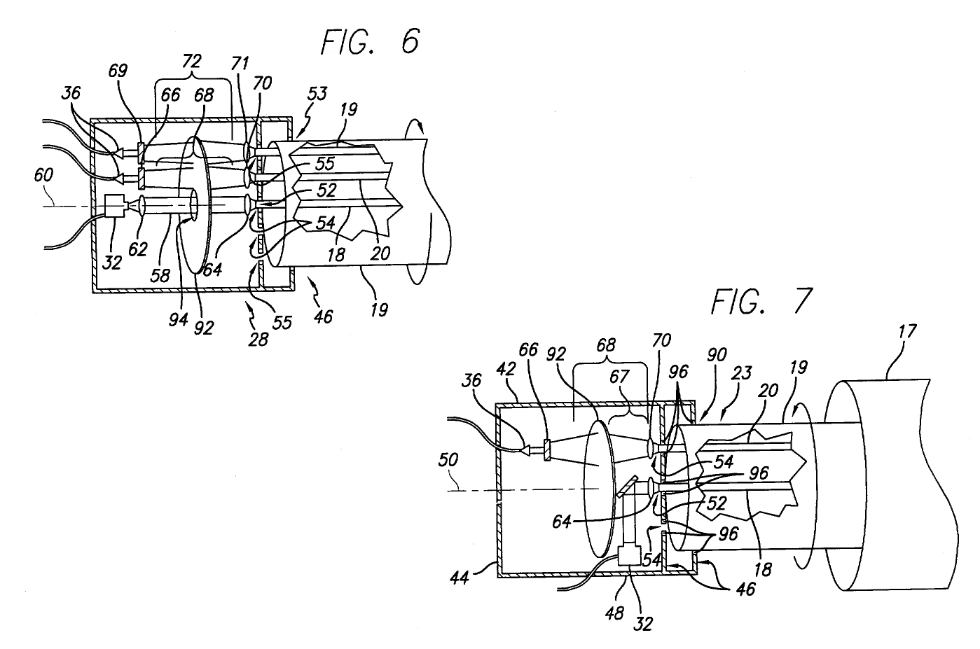

In the embodiment of FIG. 6, an additional eccentric port 55 and optical relay 71 are provided in the distal face 46. The collection beams 68 and 72 emerging from the apertures and relays form concentric nested traces on the R-S lens 92. The R-S lens 92 then directs these traces to their perspective stationary detectors 66 and 69. Analogous to the depiction and discussion of FIGS. 3 and 5, the R-S lens continuously directs the collection beams 68 and 72 onto the stationary detectors 66 and 69 as the optical relays 70 and 71 and the distal side of the collection beams rotate from 0 to 360 degrees in a circular path. This embodiment is not limited to a single additional collection beam. The embodiment would include the capacity to handle a plurality of additional collection fibers. In addition, the embodiment is not limited to only additional collection fibers. Additional delivery fibers could also be present.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An optical coupler comprising:
a housing with a distal face having an eccentric port and
a central relay for guiding a rotating second beam from the eccentric port to a fixed point on a detector.

2. The optical coupler of claim 1, further comprising:
a first optical relay configured for guiding a first beam to a central port in the distal face.

3. The optical coupler of claim 1, further comprising:
a second optical relay located between the central relay and the eccentric port for focusing the circularly rotating second beam from the rotating eccentric port to the central relay; and
wherein the central relay is configured for guiding the circularly rotating second beam from the second optical relay to the fixed point on the detector.

4. The optical coupler of claim 2, wherein the central relay comprises walls forming an aperture disposed to allow passage of the first beam.

5. The optical coupler of claim 4, wherein the housing comprises a proximal wall forming an aperture disposed to intersect the first beam.

6. The optical coupler of claim 2, further comprising a source for the first beam located between the central relay and the central port.

7. The optical coupler of claim 4, further comprising an additional optical relay disposed to guide the first beam, through the aperture, to the first optical relay

8. The optical coupler of claim 3, wherein the second optical relay rotates with the second beam.

9. The optical coupler of claim 1, wherein the central relay comprises a lens having a central axis and a focal point, the focal point being radially displaced from the central axis.

10. The optical coupler of claim 1, wherein the central relay comprises a beam-directing element having a multiplicity of zones, each zone being configured to direct a beam incident thereon to the fixed point on the detector.

11. The optical coupler of claim 1, wherein the central relay comprises a lens with a multiplicity of regions, each region having a refractive index selected to refract a beam incident thereon to the fixed point on the detector.

12. The optical coupler of claim 1, wherein the central relay comprises a lens with a multiplicity of regions, each region having a radius of curvature selected to refract a beam incident thereon to the fixed point on the detector.

13. The optical coupler of claim 3, further comprising:
a third optical relay for guiding a rotating third beam from a second rotating eccentric port on the distal face to the central relay; and
wherein the central relay is configured to guide the rotating third beam to a second fixed point on a second detector.

14. The optical coupler of claim 7, wherein the additional optical relay comprises a graded index of refraction lens seated in the central port, the graded index of refraction lens being configured to direct the first beam to the central port.

15. The optical coupler of claim 2, wherein the second optical relay comprises a collimating lens within the housing, the collimating lens being disposed to guide the second beam from the eccentric port toward the central relay.

16. The optical coupler of claim 1, wherein the stationary relay further comprises a light-directing element disposed to direct the second beam toward a peripheral wall of the housing.

17. A system for identifying vulnerable plaque, the system comprising:
a rotating catheter having a collection fiber and a delivery fiber extending therethrough;
a stationary housing, the housing having a distal face being engaged with the rotating catheter;
a first optical relay in optical communication with a central port on the distal face, the central port being in optical communication with the delivery fiber;
a second optical relay in optical communication with an eccentric port on the distal face, the eccentric port being in optical communication with the collection fiber; and
a central optical relay in optical communication with the second optical relay and a fixed point on a detector.

18. The system of claim 17, wherein the first optical relay comprises a lens disposed to receive a delivery beam passing through the central optical relay and to direct the delivery beam into the central port.

19. The system of claim 17, wherein at least one of the first, second, and central optical relays comprises a lens.

20. The system of claim 17, wherein at least one of the first, second, and central optical relays comprises a graded index of refraction lens.

21. A method for providing optical coupling to a rotating collection fiber, the method comprising:
transmitting a delivery beam to a central port in the housing, the central port being in optical communication with the delivery fiber;
receiving a rotating collection beam from a rotating eccentric port, the eccentric port being in optical communication with the collection fiber; and
providing a stationary lens to guide the rotating collection beam to a fixed point on a detector.

22. The method of claim 21, wherein transmitting the delivery beam comprises passing the delivery beam through a stationary relay.

23. The method of claim 21, wherein guiding the collection beam to the detector comprises:
relaying the collection beam from the eccentric port to a stationary relay; and
relaying the collection beam from the stationary relay to the detector.
